(19) United States
(12) Patent Application Publication
(10) Pub. No.: US 2025/0297166 A1
(43) Pub. Date: Sep. 25, 2025

(54) PROCESS TO UPGRADE AROMATIC WASTE STREAMS

(71) Applicant: Basell Poliolefine Italia S.r.l., Milan (IT)

(72) Inventors: David S. Mannel, Humble, TX (US); Sandor Nagy, Seabrook, TX (US); Barbara Kimmich, Houston, TX (US); Karl P. Rufener, Humble, TX (US); Daniel F. White, Houston, TX (US); Robert J. Casey, Kingwood, TX (US); Christopher D. Smith, Kingwood, TX (US)

(73) Assignee: Basell Poliolefine Italia S.r.l., Milan (IT)

(21) Appl. No.: 19/086,417

(22) Filed: Mar. 21, 2025

Related U.S. Application Data
(60) Provisional application No. 63/568,581, filed on Mar. 22, 2024.
(60) Provisional application No. 63/663,812, filed on Jun. 25, 2024.

(51) Int. Cl. C10G 1/10; B01J 29/06

(52) U.S. Cl. CPC C10G 1/10; B01J 29/06; C10G 2300/1003; C10G 2300/1044; C10G 2300/1096; C10G 2400/20; C10G 2400/30

(57) ABSTRACT

Disclosed are methods for upgrading heavy aromatic waste streams, including polystyrene and other related materials, to chemical feedstocks. Process embodiments include feeding heavy aromatic waste streams to: a) thermal pyrolysis followed by catalytic pyrolysis; b) thermal pyrolysis followed separation of thermal pyrolysis effluent to light and heavy streams followed by catalytic pyrolysis of the light stream; c) thermal pyrolysis followed by catalytic pyrolysis of the thermal pyrolysis effluent followed separation of the catalytic pyrolysis effluent to light and heavy streams; and d) thermal pyrolysis followed separation of thermal pyrolysis effluent to light and heavy streams followed by catalytic pyrolysis of the light stream followed by separation of the catalytic pyrolysis effluent to light and heavy streams. Each embodiment leads to production of ethylene, propylene, benzene, ethylbenzene, ethylbenzene precursors, or a combination thereof.

### CROSS-REFERENCE TO RELATED APPLICATIONS

This application claims priority of U.S. Provisional Patent Application Ser. No. 63/568,581, entitled “PROCESS TO UPGRADE AROMATIC WASTE STREAMS,” filed on Mar. 22, 2024, and U.S. Provisional Patent Application Ser. No. 63/663,812, entitled “CATALYTIC CRACKING OF POSM FUEL UPGRADE BOTTOMS (“IRFO”),” filed on Jun. 25, 2024, the contents of which are incorporated by reference herein in their entirety.

### FIELD OF THE INVENTION

This disclosure relates generally to the field of chemical recycling and more specifically to processes for recycling and converting heavy aromatic waste and/or fuel oil streams, including polystyrene and other related materials, back to chemical feedstocks.

### BACKGROUND OF THE INVENTION

The production of styrene and ethylbenzene is a critical aspect of the chemical industry, with applications spanning numerous products, including plastics, resins, and rubber. The Propylene Oxide/Styrene Monomer (POSM) process is a well-known technology for producing styrene and propylene oxide. However, the reliance on non-renewable resources and the generation of by-products and waste streams present environmental and economic challenges.

Polystyrene waste, a significant environmental concern due to its non-biodegradability, has traditionally been addressed through mechanical recycling processes. However, these processes are limited in their ability to achieve complete chemical circularity and often result in downcycling of the material. Furthermore, heavy aromatic waste streams, such as residual fuel oils (RFOs) and heavy aromatic solvents (HAS), are by-products of various chemical processes, including the POSM process itself. These waste streams are often underutilized and pose disposal and environmental issues.

There is a need for improved processes to recycle these waste streams back into valuable chemical feedstocks, thereby reducing reliance on virgin raw materials, enhancing the sustainability of chemical processes, and contributing to the circular economy. Ideally, such improved processes could be implemented in existing facilities by retrofitting or built into new facilities employing commonly used equipment and familiar techniques.

### SUMMARY OF THE INVENTION

In some embodiments, a process to upgrade a heavy aromatic waste stream to chemical feedstocks comprises subjecting the heavy aromatic waste stream to thermal pyrolysis conditions in a first reaction zone to produce a first intermediate product and char. Optionally, the first intermediate product is subjected to separation conditions to produce a second intermediate product and a first residual fraction. The first intermediate product or the second intermediate product is then subjected to catalytic pyrolysis conditions in a second reaction zone to produce a third intermediate product comprising styrene, benzene, ethylene, propylene, or a combination thereof. Optionally, the third intermediate product is subjected to separation conditions to produce a fourth intermediate product comprising styrene, benzene, ethylene, propylene, or a combination thereof and a second residual fraction.

The foregoing has outlined rather broadly the features and technical advantages of the present invention in order that the detailed description of the invention that follows may be better understood. Additional features and advantages of the invention will be described hereinafter, which form the subject matter of the claims of the invention. It should be appreciated by those skilled in the art that the conception and specific embodiments disclosed may be readily utilized as a basis for modifying or designing other catalyst compositions and/or processes for carrying out the same purposes of the present invention. It should also be realized by those skilled in the art that such equivalent constructions do not depart from the spirit and scope of the invention as set forth in the appended claims. The novel features which are believed to be characteristic of the invention, both as to its compositions and processes, together with further objects and advantages will be better understood from the following description.

### BRIEF DESCRIPTION OF THE FIGURES

The claimed subject matter may be understood by reference to the following description taken in conjunction with the accompanying drawings, in which like reference numerals identify like elements, and in which:

FIG. 1 is a simplified flow diagram illustrating thermal pyrolysis of heavy aromatic waste feed to produce a first intermediate product followed by catalytic pyrolysis of the first intermediate product to form a second intermediate product, according to embodiments of the disclosure;

FIG. 2 is a simplified flow diagram illustrating thermal pyrolysis of heavy aromatic waste feed to produce a first intermediate product, and separation of the first intermediate product into a second intermediate product and a first residual fraction, followed by catalytic pyrolysis of the second intermediate product to form a third intermediate product, according to embodiments of the disclosure;

FIG. 3 is a simplified flow diagram illustrating thermal pyrolysis of heavy aromatic waste feed to produce a first intermediate product, followed by catalytic pyrolysis of the first intermediate product to form a second intermediate product, and separation of the second intermediate product into a third intermediate product and a first residual fraction, according to embodiments of the disclosure;

FIG. 4 is a simplified flow diagram illustrating thermal pyrolysis of heavy aromatic waste feed to produce a first intermediate product, and separation of the first intermediate product into a second intermediate product and a first residual fraction, followed by catalytic pyrolysis of the second intermediate product to form a third intermediate product, and separation of the third intermediate product into a fourth intermediate product and a second residual fraction, according to embodiments of the disclosure; and

FIG. 5A and FIG. 5B show a collection of various spectra showing the composition of a fuel oil, according to an embodiment of the disclosure (FIG. 5A), and a series of three MS-GC spectra representing the composition of the catalytically cracked fuel oil (FIG. 5B).

While the disclosed process and composition are susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the subject matter claimed below will now be disclosed. In the interest of clarity, some features of some actual implementations may not be described in this specification. It will be appreciated that in the development of any such actual embodiments, numerous implementation-specific decisions must be made to achieve the developer's specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

### Definitions

As used herein, “a” or “an” when used in conjunction with the term “comprising” in the claims or the specification means one or more than one, unless the context dictates otherwise.

As used herein, “about” means the stated value plus or minus the margin of error of measurement or plus or minus 10% if no process of measurement is indicated.

As used herein, “char” refers to coke, a carbon-containing solid, that accumulates on the catalyst particles during pyrolysis.

As used herein, “consisting essentially of” excludes additional material elements, but allows the inclusions of non-material elements that do not substantially change the nature of the invention.

As used herein, “consisting of” is closed and excludes all additional elements.

As used herein, “conversion” is used to denote the percentage of a component fed which disappears across a reactor.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used herein, “reaction zone” refers to a chamber sufficiently enclosed to maintain selected operating conditions within the chamber to produce a desired reaction, such as a pyrolysis reaction zone, a steam cracking reaction zone, a catalytic cracking reaction zone, or a hydrogenation reaction zone. In some embodiments, each reaction zone can be a separate reactor. In some embodiments, a single vessel can contain a plurality of reaction zones.

As used herein, “separation conditions” or “separation section” means facilities including distillation, absorption, adsorption, membrane separation, cryogenic distillation, extractive distillation, azeotropic distillation, steam distillation, molecular sieves, liquid-liquid extraction (solvent extraction), decantation, centrifugation, or a combination thereof as required to recover specific materials from intermediate products and/or reaction products disclosed herein. Such embodiments would include common equipment associated with the forgoing separation processes, including, but not limited to, columns, drums, vessels, heat exchangers, pumps, valves, reflux loops, and the like, the descriptions of which are omitted herein for simplicity. Where intermediate products and/or reaction products disclosed herein are defined as comprising multiple products (e.g., benzene, ethylene, propylene, or a combination thereof), it is intended that “separation conditions” or “separation section” includes the functionality to recover each of those products separately at a desired purity (e.g., 99 wt % benzene, 99.5 wt % ethylene, 97 wt % propylene, etc.) as required by the disposition of the product as a feed to various locations of other processes, such as, but not limited to, POSM and/or SM.

As used herein, “waste stream” is a type of feed stream comprising material that has been discarded as no longer useful, including but not limited to, post-consumer and post-industrial waste.

As used herein, “zeolite” refers to an aluminosilicate mineral with a microporous structure. Zeolites are, in one aspect, useful as catalysts for the processes disclosed herein. Zeolites can occur naturally or can be produced industrially.

All concentrations herein are by weight percent (“wt %”) unless otherwise specified.

The following abbreviations are used herein:

| Abbreviation | Term |
|---|---|
| ECR | ethylene cracking residue |
| HAS | heavy aromatic solvent |
| PAH | polycyclic aromatic hydrocarbons |
| PFO | pyrolysis fuel oil |
| POSM | Propylene oxide/styrene monomer production process |
| RFO | residual fuel oils |
| SM | Styrene monomer or process to produce styrene monomer, depending |
| | on the context |
| WHSV | weight hourly space velocity |

### Heavy Aromatic Waste

The present disclosure provides a process for upgrading aromatic waste, including polystyrene, into light olefins (e.g., ethylene and/or propylene), ethylbenzene, and/or ethylbenzene precursors useful as feedstocks to petrochemical processes, including, but not limited to, the propylene oxide/styrene monomer process and dehydrogenation, oxidative or non-oxidative, of ethylbenzene to produce styrene. Heavy aromatic waste streams include, but are not limited to, polystyrene pyrolysis oil, styrene, polystyrene pyrolysis oil heavies, heavy residual fuel oils (RFO), heavy aromatic solvent (HAS), pyrolysis fuel oil (PFO), and ethylene cracking residue (ECR). Heavy aromatic waste streams to be treated by the processes disclosed herein can be any one of the foregoing materials or any mixture of two or more of the foregoing materials.

Polystyrene pyrolysis involves the thermal degradation of polystyrene, a common thermoplastic material, in the absence of oxygen. This process breaks down the long polymer chains of polystyrene back into shorter hydrocarbon chains and styrene monomer, along with a range of other hydrocarbons. The products of polystyrene pyrolysis can be broadly categorized into two groups: polystyrene pyrolysis oil and polystyrene pyrolysis oil heavies. Each of these categories has distinct characteristics and potential applications. Polystyrene pyrolysis oil primarily consists of styrene monomer, which can be recovered and purified for reuse in the production of new polystyrene products or other styrenic polymers. Besides styrene, the oil contains a mixture of other aromatic hydrocarbons such as toluene, ethylbenzene, and benzene, as well as aliphatic hydrocarbons. The exact composition of the pyrolysis oil can vary based on the pyrolysis conditions such as temperature, heating rate, and the presence of catalysts. Polystyrene pyrolysis oil heavies consist of the heavier, more complex hydrocarbons that are produced during the pyrolysis process. These compounds are higher in molecular weight compared to the main fraction of pyrolysis oil and often include polycyclic aromatic hydrocarbons (PAH), along with various oligomers formed by partial recombination of degradation products. Pyrolysis of polystyrene presents an opportunity for recycling a plastic that is otherwise difficult to process through mechanical recycling methods. By converting waste polystyrene into valuable chemicals and fuels, pyrolysis can reduce landfilling and incineration, contributing to circular economy initiatives.

During the POSM process, various by-products are generated, including residual fuel oils (RFO) rich in aromatics. These RFOs are complex mixtures containing high molecular weight hydrocarbons, predominantly aromatics, along with aliphatics and small amounts of olefins. The aromatic content gives these oils their distinct characteristics, including high density and a high calorific value. The exact composition of these oils can vary depending on the specifics of the POSM process and the feedstocks used. These oils have high boiling points due to the presence of large, complex hydrocarbon molecules. The high aromatic content contributes to a higher density and viscosity compared to lighter fuel oils. While these oils are rich in energy content, their high aromatic content may affect their combustion characteristics. Depending on the feedstock and process conditions, the sulfur content can vary, potentially requiring desulfurization treatments for certain uses. Such RFOs have utility as components in fuel oil and asphalt blending but incur some safety, environmental, and regulatory considerations due to high aromatics content and/or emissions such as NOx, SOx, and particulate matter.

Benzene is typically converted to ethylbenzene by reacting benzene with ethylene in an alkylation process. Heavy aromatic solvents (HAS) can be produced as by-products of the alkylation process. These heavy aromatic solvents are generally composed of higher molecular weight aromatic compounds that form through side reactions during the alkylation process. A common HAS produced in this context is diethylbenzene (DEB), which consists of three isomers: ortho-, meta-, and para-diethylbenzene. Diethylbenzene forms when an ethyl group is added to ethylbenzene in a subsequent alkylation reaction, essentially representing an over-alkylation of benzene. Although DEB has some direct uses, it would be desirable to break down and convert these heavy molecules into basic monomers suitable as feedstocks to a variety of petrochemical processes, including, but not limited to, POSM and EB dehydrogenation to SM.

In steam cracking processes, complex mixtures of by-products are generated in addition to producing olefins (e.g., ethylene, propylene). Such by-products include heavy aromatic-rich higher molecular weight hydrocarbons such as pyrolysis fuel oil (PFO) and ethylene cracking residue (ECR). PFO is a complex mixture that includes heavy aromatics, asphaltenes, and other hydrocarbons produced during the thermal cracking of naphtha or gas oils. Its exact composition varies depending on the feedstock and the operating conditions of the cracker but is characterized by its high content of polycyclic aromatic hydrocarbons (PAHs). ECR is the residue left from the steam cracking process, particularly when heavier feedstocks are used. It is rich in heavy aromatics, tar, and coke precursors. Like PFO, its specific composition depends on the feedstock and process conditions. Similarly to RFOs, PFO and ECR have utility as components in fuel oil but incur some safety, environmental, and regulatory considerations due to high aromatics content and/or emissions such as NOx, SOx, and particulate matter.

The present disclosure provides embodiments of processes to upgrade heavy aromatic waste streams, including polystyrene and other related materials, to chemical feedstocks. In some embodiments, the heavy aromatic waste stream comprises polystyrene pyrolysis oil, styrene, polystyrene pyrolysis oil heavies, heavy residual fuel oils (RFO), heavy aromatic solvent (HAS), pyrolysis fuel oil (PFO), ethylene cracking residue (ECR), or a combination thereof. In some embodiments, the heavy aromatic waste stream comprises styrene in an amount greater than or equal to 30 wt %, greater than or equal to 40 wt %, greater than or equal to 50 wt %, or greater than or equal to 60 wt %, based on the total weight of the heavy aromatic waste stream.

In some embodiments, the present disclosure provides a method for converting a fuel oil to propylene comprising: (a) providing a fuel oil comprising a plurality of aromatic compounds comprising: (i) from about 1.0 wt % to about 2.0 wt % ethylbenzene; and (ii) from about 1.0 wt % to about 2.0 wt % styrene, wherein the fuel oil comprises no more than 1.0 wt % benzene and 0.2 wt % propylene; and (b) catalytically cracking the fuel oil in the presence of a catalyst to produce a cracked product comprising: (iii) from about 10 wt % to about 19 wt % propylene; and (iv) from about 36 wt % to about 50 wt % benzene.

### Process Comprising Thermal/Catalytic Pyrolysis

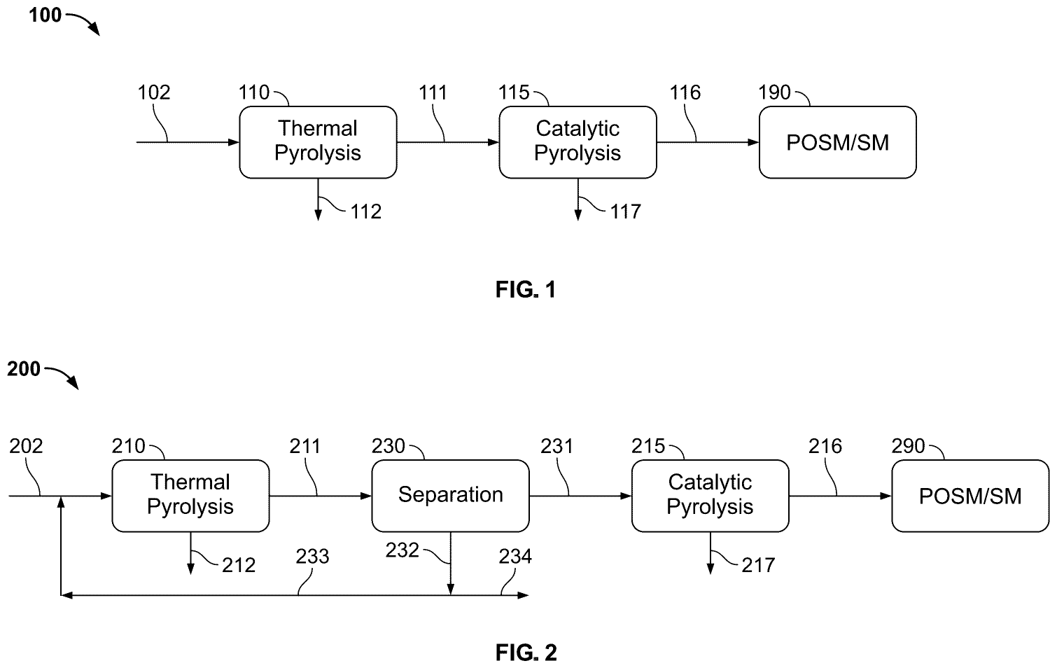

FIG. 1-FIG. 4 show various embodiments of a process for upgrading heavy aromatic streams as described above. Some embodiments include process steps for separation of one or more intermediate product stream before progressing to the next process step.

FIG. 1 shows a process 100 to upgrade a heavy aromatic waste stream 102 into chemical feedstocks. The heavy aromatic waste stream 102 has a first styrene content, a first benzene content, a first ethylene content, a first propylene content, or a combination thereof. The process 100 comprises subjecting the heavy aromatic waste stream 102 to thermal pyrolysis conditions in a first reaction zone 110 to produce a first reaction product 111 and char 112. The process conditions and catalyst selection are optimized to achieve a desired product composition. In some embodiments, the first reaction product 111 has a second styrene content less than the first styrene content, a second benzene content less than the first benzene content, a second ethylene content less than the first styrene content, a second propylene content less than the first propylene content, or a combination thereof. The thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

The first reaction product 111 is then fed to a second reaction zone 115, wherein it is subjected to catalytic pyrolysis to produce a second reaction product 116 and char 117. The process conditions and catalyst selection are optimized to achieve a desired product composition. In some embodiments, the second reaction product 116 has a third styrene content less than the second styrene content, a third benzene content less than the second benzene content, a third ethylene content less than the second styrene content, a third propylene content less than the second propylene content, or a combination thereof. The catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

In some embodiments, catalytic cracking the fuel oil occurs at a temperature greater than about 380° C. In some embodiments, catalytic cracking the fuel oil occurs at a temperature greater than about 480° C. In some embodiments, catalytic cracking the fuel oil occurs at a temperature greater than about 580° C. In some embodiments, catalytic cracking the fuel oil occurs at a temperature in the range of from about 350° C. to about 650° C. In some embodiments, catalytic cracking the fuel oil occurs at a temperature in the range of from about 350° C. to about 450° C. In some embodiments, catalytic cracking the fuel oil occurs at a temperature in the range of from about 450° C. to about 550° C. In some embodiments, catalytic cracking the fuel oil occurs at a temperature in the range of from about 550° C. to about 650° C. In some embodiments, the catalyst is a zeolite. In some embodiments, the catalyst is a H-ZSM-5 zeolite. In some embodiments, the fuel oil is obtained from a propylene oxide/styrene monomer (POSM) production process.

In some embodiments, the second reaction zone 115 further comprises a medium or large pore zeolite or molecular sieve. The medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

The second reaction product 116 is then fed to a POSM/SM process 190, wherein POSM/SM means the POSM process and/or the SM process. SM process means the direct dehydrogenation of EB to form styrene monomer. The second reaction product 116 comprises one or more of styrene, benzene, ethylene, and propylene. As part of POSM/SM process 190, one or more of styrene, benzene, ethylene, and propylene is recovered from second reaction product 116, wherein the purity of the recovered styrene, benzene, ethylene, and propylene is suitable for direct or indirect addition to the POSM and/or the SM process. Indirect addition means that one or more additional processing steps can be performed on the recovered stream to make it suitable for addition to the POSM and/or the SM process. The specific components in the heavy aromatic waste stream 102, the specific thermal pyrolysis conditions in the first reaction zone 110, and the specific catalytic pyrolysis conditions in the second reaction zone 115 will result in different amounts of styrene, benzene, ethylene, and/or propylene in the second reaction product 116.

In some embodiments, styrene is recovered from the second reaction product 116. The styrene is hydrogenated to form EB. Examples of hydrogenation catalysts include any commercial hydrogenation catalysts known in the art such as NiMo, CoMo Ni, Pd, Pt, Cu, and the like. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, benzene is recovered from the second reaction product 116. The benzene is reacted with ethylene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, ethylene is recovered from the second reaction product 116. The ethylene is reacted with benzene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, propylene is recovered from the second reaction product 116. The propylene is added to the POSM process, wherein it is reacted with ethylbenzene hydroperoxide to form propylene oxide and 1-phenyl ethanol. The 1-phenyl ethanol is then dehydrated to produce styrene monomer.

Referring to FIG. 1, in some embodiments, the first reaction product 111 is sent directly to the POSM and/or SM process 190. In some embodiments, the heavy aromatic waste 102 is sent directly to catalytic pyrolysis in the second reaction zone 115.

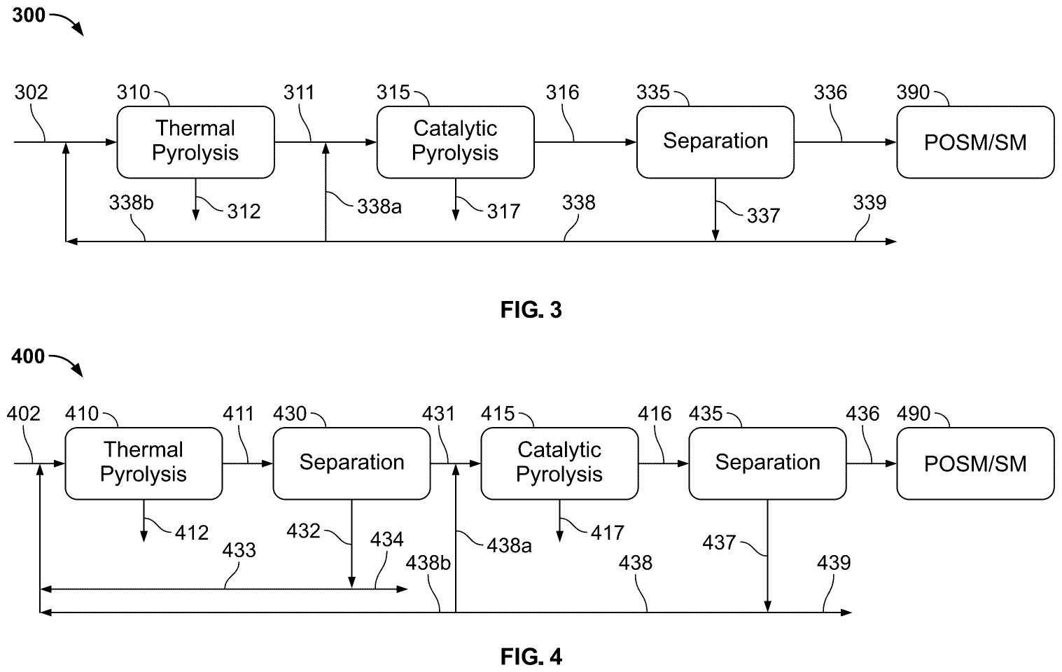

FIG. 2 shows a process 200 to upgrade a heavy aromatic waste stream 202 into chemical feedstocks. The heavy aromatic waste stream 202 has a first styrene content, a first benzene content, a first ethylene content, a first propylene content, or a combination thereof. The process 200 comprises subjecting the heavy aromatic waste stream 202 to thermal pyrolysis conditions in a first reaction zone 210 to produce a first intermediate product 211 and char 212. The process conditions and catalyst selection are optimized to achieve a desired product composition. In some embodiments, the first intermediate product has a second styrene content less than the first styrene content, a second benzene content less than the first benzene content, a second ethylene content less than the first styrene content, a second propylene content less than the first propylene content, or a combination thereof. The thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

The first intermediate product 211 is subjected to separation conditions 230 sufficient to produce a second intermediate product 231 and a first residual fraction 232. The second intermediate product has a third styrene content less than the second styrene content, a third benzene content less than the second benzene content, a third ethylene content less than the second styrene content, a third propylene content less than the second propylene content, or a combination thereof.

The second intermediate product 231 is then fed to a second reaction zone 215, wherein it is subjected to catalytic pyrolysis to produce a third intermediate product 216 and char 217. The third intermediate product 216 has a fourth styrene content less than the third styrene content, a fourth benzene content less than the third benzene content, a fourth ethylene content less than the third styrene content, a fourth propylene content less than the third propylene content, or a combination thereof. The catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

In some embodiments, catalytic cracking the fuel oil occurs at a temperature greater than about 380° C. In some embodiments, catalytic cracking the fuel oil occurs at a temperature greater than about 480° C. In some embodiments, catalytic cracking the fuel oil occurs at a temperature greater than about 580° C. In some embodiments, catalytic cracking the fuel oil occurs at a temperature in the range of from about 350° C. to about 650° C. In some embodiments, catalytic cracking the fuel oil occurs at a temperature in the range of from about 350° C. to about 450° C. In some embodiments, catalytic cracking the fuel oil occurs at a temperature in the range of from about 450° C. to about 550° C. In some embodiments, catalytic cracking the fuel oil occurs at a temperature in the range of from about 550° C. to about 650° C. In some embodiments, the catalyst is a zeolite. In some embodiments, the catalyst is a H-ZSM-5 zeolite. In some embodiments, the fuel oil is obtained from a propylene oxide/styrene monomer (POSM) production process.

In some embodiments, the second reaction zone 215 further comprises a medium or large pore zeolite or molecular sieve. The medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

The second reaction product 216 is then fed to a POSM/SM process 290, wherein POSM/SM means the POSM process and/or the SM process. SM process means the direct dehydrogenation of EB to form styrene monomer. The second reaction product 216 comprises one or more of styrene, benzene, ethylene, and propylene. As part of POSM/SM process 290, one or more of styrene, benzene, ethylene, and propylene is recovered from second reaction product 216, wherein the purity of the recovered styrene, benzene, ethylene, and propylene is suitable for direct or indirect addition to the POSM and/or the SM process. Indirect addition means that one or more additional processing steps can be performed on the recovered stream to make it suitable for addition to the POSM and/or the SM process. The specific components in the heavy aromatic waste stream 202, the specific thermal pyrolysis conditions in the first reaction zone 210, and the specific catalytic pyrolysis conditions in the second reaction zone 215 will result in different amounts of styrene, benzene, ethylene, and/or propylene in the second reaction product 216.

In some embodiments, styrene is recovered from the second reaction product 216. The styrene is hydrogenated to form EB. Examples of hydrogenation catalysts include any commercial hydrogenation catalysts known in the art such as NiMo, CoMo, Ni, Pd, Pt, Cu, and the like. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, benzene is recovered from the second reaction product 216. The benzene is reacted with ethylene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, ethylene is recovered from the second reaction product 216. The ethylene is reacted with benzene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, propylene is recovered from the second reaction product 216. The propylene is added to the POSM process, wherein it is reacted with ethylbenzene hydroperoxide to form propylene oxide and 1-phenyl ethanol. The 1-phenyl ethanol is then dehydrated to produce styrene monomer.

First residual fraction 232 is added to the first reaction zone 210 as additional feed via stream 233, sent outside the process for addition processing via stream 234, or a combination thereof.

Referring to FIG. 2, in some embodiments, the second intermediate product 231 is sent directly to the POSM and/or SM process 290.

FIG. 3 shows a process 300 to upgrade a heavy aromatic waste stream 302 into chemical feedstocks. The heavy aromatic waste stream 302 has a first styrene content, a first benzene content, a first ethylene content, a first propylene content, or a combination thereof. The process 300 comprises subjecting the heavy aromatic waste stream 302 to thermal pyrolysis conditions in a first intermediate zone 310 to produce a first intermediate product 311 and char 312. The process conditions and catalyst selection are optimized to achieve a desired product composition. In some embodiments, the first intermediate product has a second styrene content less than the first styrene content, a second benzene content less than the first benzene content, a second ethylene content less than the first styrene content, a second propylene content less than the first propylene content, or a combination thereof. The thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

The first reaction product 311 is then fed to a second reaction zone 315, wherein it is subjected to catalytic pyrolysis to produce a second reaction product 316 and char 317. The second reaction product 316 has a third styrene content less than the second styrene content, a third benzene content less than the second benzene content, a third ethylene content less than the second styrene content, a third propylene content less than the second propylene content, or a combination thereof. The catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

In some embodiments, catalytic cracking the fuel oil occurs at a temperature greater than about 380° C. In some embodiments, catalytic cracking the fuel oil occurs at a temperature greater than about 480° C. In some embodiments, catalytic cracking the fuel oil occurs at a temperature greater than about 580° C. In some embodiments, catalytic cracking the fuel oil occurs at a temperature in the range of from about 350° C. to about 650° C. In some embodiments, catalytic cracking the fuel oil occurs at a temperature in the range of from about 350° C. to about 450° C. In some embodiments, catalytic cracking the fuel oil occurs at a temperature in the range of from about 450° C. to about 550° C. In some embodiments, catalytic cracking the fuel oil occurs at a temperature in the range of from about 550° C. to about 650° C. In some embodiments, the catalyst is a zeolite. In some embodiments, the catalyst is a H-ZSM-5 zeolite. In some embodiments, the fuel oil is obtained from a propylene oxide/styrene monomer (POSM) production process.

In some embodiments, the second reaction product 316 is subjected to separation conditions 335 sufficient to produce a third reaction product 336 and a first residual fraction 337. The third reaction product 336 has a fourth styrene content less than the third styrene content, a fourth benzene content less than the third benzene content, a fourth ethylene content less than the third styrene content, a fourth propylene content less than the third propylene content, or a combination thereof.

The second reaction zone 315 further comprises a medium or large pore zeolite or molecular sieve. The medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

The third reaction product 316 is then fed to a POSM/SM process 390, wherein POSM/SM means the POSM process and/or the SM process. SM process means the direct dehydrogenation of EB to form styrene monomer. The third reaction product 316 comprises one or more of styrene, benzene, ethylene, and propylene. As part of POSM/SM process 390, one or more of styrene, benzene, ethylene, and propylene is recovered from third reaction product 316, wherein the purity of the recovered styrene, benzene, ethylene, and propylene is suitable for direct or indirect addition to the POSM and/or the SM process. Indirect addition means that one or more additional processing steps can be performed on the recovered stream to make it suitable for addition to the POSM and/or the SM process. The specific components in the heavy aromatic waste stream 302, the specific thermal pyrolysis conditions in the first reaction zone 310, and the specific catalytic pyrolysis conditions in the second reaction zone 315 will result in different amounts of styrene, benzene, ethylene, and/or propylene in third reaction product 316.

In some embodiments, styrene is recovered from the third reaction product 316. The styrene is hydrogenated to form EB. Examples of hydrogenation catalysts include any commercial hydrogenation catalysts known in the art such as NiMo, CoMo, Ni, Pd, Pt, Cu, and the like. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, benzene is recovered from the third reaction product 316. The benzene is reacted with ethylene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, ethylene is recovered from the third reaction product 316. The ethylene is reacted with benzene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, propylene is recovered from the third reaction product 316. The propylene is added to the POSM process, wherein it is reacted with ethylbenzene hydroperoxide to form propylene oxide and 1-phenyl ethanol. The 1-phenyl ethanol is then dehydrated to produce styrene monomer.

First residual fraction 352 is added to the first reaction zone 310 as additional feed via stream 338, 338b, added to the second reaction zone 315 as additional feed via stream 338, 338a, sent outside the process for addition processing via stream 339, or a combination thereof.

Referring to FIG. 3, in some embodiments, the heavy aromatic waste 302 sent directly to catalytic pyrolysis in the second reaction zone 315.

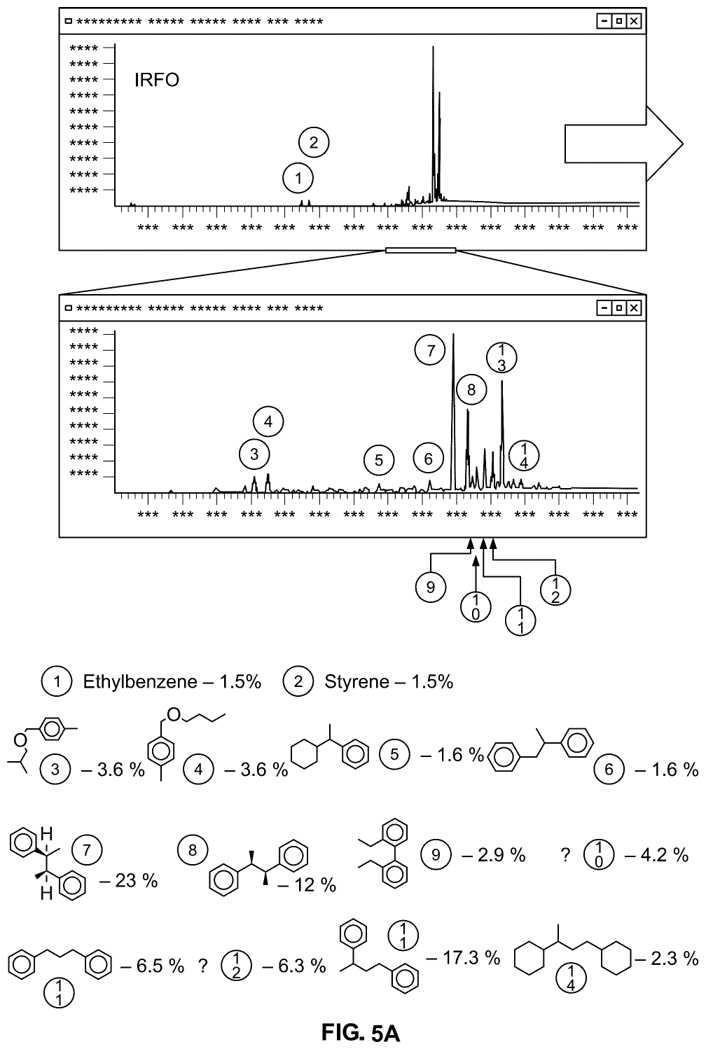

FIG. 4 shows a process 400 to upgrade a heavy aromatic waste stream 402 into chemical feedstocks. The heavy aromatic waste stream 402 has a first styrene content, a first benzene content, a first ethylene content, a first propylene content, or a combination thereof. The process 400 comprises subjecting the heavy aromatic waste stream 402 to thermal pyrolysis conditions in a first reaction zone 410 to produce a first reaction product 411 and char 412. The process conditions and catalyst selection are optimized to achieve a desired product composition. In some embodiments, the first intermediate product has a second styrene content less than the first styrene content, a second benzene content less than the first benzene content, a second ethylene content less than the first styrene content, a second propylene content less than the first propylene content, or a combination thereof. The thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

The first intermediate product 411 is subjected to separation conditions 430 sufficient to produce a second intermediate product 431 and a first residual fraction 432. The second intermediate product has a third styrene content less than the second styrene content, a third benzene content less than the second benzene content, a third ethylene content less than the second styrene content, a third propylene content less than the second propylene content, or a combination thereof.

The second intermediate product 431 is then fed to a second reaction zone 415, wherein it is subjected to catalytic pyrolysis to produce a third intermediate product 416 and char 417. The third intermediate product 416 has a fourth styrene content less than the third styrene content, a fourth benzene content less than the third benzene content, a fourth ethylene content less than the third styrene content, a fourth propylene content less than the third propylene content, or a combination thereof. The catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

In some embodiments, catalytic cracking the fuel oil occurs at a temperature greater than about 380° C. In some embodiments, catalytic cracking the fuel oil occurs at a temperature greater than about 480° C. In some embodiments, catalytic cracking the fuel oil occurs at a temperature greater than about 580° C. In some embodiments, catalytic cracking the fuel oil occurs at a temperature in the range of from about 350° C. to about 650° C. In some embodiments, catalytic cracking the fuel oil occurs at a temperature in the range of from about 350° C. to about 450° C. In some embodiments, catalytic cracking the fuel oil occurs at a temperature in the range of from about 450° C. to about 550° C. In some embodiments, catalytic cracking the fuel oil occurs at a temperature in the range of from about 550° C. to about 650° C. In some embodiments, the catalyst is a zeolite. In some embodiments, the catalyst is a H-ZSM-5 zeolite. In some embodiments, the fuel oil is obtained from a propylene oxide/styrene monomer (POSM) production process.

In some embodiments, the second reaction zone 415 further comprises a medium or large pore zeolite or molecular sieve. The medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

The third intermediate product 416 is subjected to separation conditions 435 sufficient to produce a fourth intermediate product 436 and a first residual fraction 437. The fourth intermediate product 436 has a fifth styrene content less than the fourth styrene content, a fifth benzene content less than the fourth benzene content, a fifth ethylene content less than the fourth styrene content, a fifth propylene content less than the fourth propylene content, or a combination thereof.

The fourth intermediate product 436 is then fed to a POSM/SM process 490, wherein POSM/SM means the POSM process and/or the SM process. SM process means the direct dehydrogenation of EB to form styrene monomer. The fourth intermediate product 436 comprises one or more of styrene, benzene, ethylene, and propylene. As part of POSM/SM process 490, one or more of styrene, benzene, ethylene, and propylene is recovered from fourth intermediate product 436, wherein the purity of the recovered styrene, benzene, ethylene, and propylene is suitable for direct or indirect addition to the POSM and/or the SM process. Indirect addition means that one or more additional processing steps can be performed on the recovered stream to make it suitable for addition to the POSM and/or the SM process. The specific components in the heavy aromatic waste stream 402, the specific thermal pyrolysis conditions in the first reaction zone 410, and the specific catalytic pyrolysis conditions in the second reaction zone 415 will result in different amounts of styrene, benzene, ethylene, and/or propylene in the fourth intermediate product 436.

In some embodiments, styrene is recovered from the fourth intermediate product 436. The styrene is hydrogenated to form EB. Examples of hydrogenation catalysts include any commercial hydrogenation catalysts known in the art such as NiMo, CoMo, Ni, Pd, Pt, Cu, and the like. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, benzene is recovered from the fourth intermediate product 436. The benzene is reacted with ethylene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, ethylene is recovered from the fourth intermediate product 436. The ethylene is reacted with benzene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, propylene is recovered from the fourth intermediate product 436. The propylene is added to the POSM process, wherein it is reacted with ethylbenzene hydroperoxide to form propylene oxide and 1-phenyl ethanol. The 1-phenyl ethanol is then dehydrated to produce styrene monomer.

First residual fraction 432 is added to the first reaction zone 410 as additional feed via stream 433, sent outside the process for addition processing via stream 434, or a combination thereof.

Second residual fraction 437 is added to the first reaction zone 410 as additional feed via stream 438, 438b, added to the second reaction zone 415 as additional feed via stream 438, 438a, sent outside the process for addition processing via stream 439, or a combination thereof.

### Certain Embodiments

Embodiment A1. A process to upgrade a heavy aromatic waste stream to chemical feedstocks, the process comprising:
a) subjecting the heavy aromatic waste stream to thermal pyrolysis conditions in a first reaction zone to produce a first intermediate product;
b) optionally subjecting the first intermediate product to separation conditions to produce a second intermediate product and a first residual fraction;
c) subjecting the first intermediate product or the second intermediate product to catalytic pyrolysis conditions in a second reaction zone to produce a third intermediate product comprising styrene, benzene, ethylene, propylene, or a combination thereof; and
d) optionally subjecting the third intermediate product to separation conditions to produce a fourth intermediate product comprising styrene, benzene, ethylene, propylene, or a combination thereof and a second residual fraction.

Embodiment A2. The process of Embodiment A1, wherein:
a) the heavy aromatic waste stream has a first styrene content;
b) the first intermediate product has a second styrene content, and the second styrene content is greater than the first styrene content;
c) the second intermediate product has a third styrene content, and the third styrene content is greater than the second styrene content;
d) the third intermediate product has a fourth styrene content, and the fourth styrene content is greater than the third styrene content; and
e) the fourth intermediate product has a fifth styrene content, and the fifth styrene content is greater than the fourth styrene content.

Embodiment A3. The process of Embodiment A1 or A2, wherein:
a) the heavy aromatic waste stream has a first benzene content;
b) the first intermediate product has a second benzene content, and the second benzene content is greater than the first benzene content;
c) the second intermediate product has a third benzene content, and the third benzene content is greater than the second benzene content;
d) the third intermediate product has a fourth benzene content, and the fourth benzene content is greater than the third benzene content; and
e) the fourth intermediate product has a fifth benzene content, and the fifth benzene content is greater than the fourth benzene content.

Embodiment A4. The process any one of Embodiments A1-A3, wherein:
a) the heavy aromatic waste stream has a first ethylene content;
b) the first intermediate product has a second ethylene content, and the second ethylene content is greater than the first ethylene content;
c) the second intermediate product has a third ethylene content, and the third ethylene content is greater than the second ethylene content;
d) the third intermediate product has a fourth ethylene content, and the fourth ethylene content is greater than the third ethylene content; and
e) the fourth intermediate product has a fifth ethylene content, and the fifth ethylene content is greater than the fourth ethylene content.

Embodiment A5. The process any one of Embodiments A1-A4, wherein:
a) the heavy aromatic waste stream has a first propylene content;
b) the first intermediate product has a second propylene content, and the second propylene content is greater than the first propylene content;
c) the second intermediate product has a third propylene content, and the third propylene content is greater than the second propylene content;
d) the third intermediate product has a fourth propylene content, and the fourth propylene content is greater than the third propylene content; and
e) the fourth intermediate product has a fifth propylene content, and the fifth propylene content is greater than the fourth propylene content.

Embodiment A6. The process any one of Embodiments A1-A5, wherein the thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

Embodiment A7. The process any one of Embodiments A1-A6, wherein the catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

Embodiment A8. The process any one of Embodiments A1-A7, wherein the second reaction zone comprises a medium or large pore zeolite or molecular sieve.

Embodiment A9. The process of Embodiment A8, wherein the medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

Embodiment A10. The process any one of Embodiments A1-A9, further comprising:
a) recovering the styrene from the third intermediate product or the fourth intermediate product; and
b) hydrogenating the styrene to form ethylbenzene.

Embodiment A11. The process any one of Embodiments A1-A10, further comprising:
a) oxidizing at least a portion of the ethylbenzene to form ethylbenzene hydroperoxide, catalytically reacting the ethylbenzene hydroperoxide with propylene to form propylene oxide and 1-phenyl ethanol, and dehydrating the 1-phenyl ethanol to produce styrene monomer;
b) dehydrogenating at least a portion of the ethylbenzene to produce styrene monomer; or
c) a combination thereof.

Embodiment A12. The process any one of Embodiments A1-A11, further comprising:
a) recovering the benzene from the third intermediate product or the fourth intermediate product; and
b) reacting the benzene with ethylene to form ethylbenzene.

Embodiment A13. The process of Embodiment A12, further comprising:
a) oxidizing at least a portion of the ethylbenzene to form ethylbenzene hydroperoxide, catalytically reacting the ethylbenzene hydroperoxide with propylene to form propylene oxide and 1-phenyl ethanol, and dehydrating the 1-phenyl ethanol to produce styrene monomer;
b) dehydrogenating at least a portion of the ethylbenzene to produce styrene monomer; or
c) a combination thereof.

Embodiment A14. The process any one of Embodiments A1-A13, further comprising:
a) recovering the ethylene from the third intermediate product or the fourth intermediate product; and
b) reacting the ethylene with benzene to form ethylbenzene.

Embodiment A15. The process of Embodiment A14, further comprising:
a) oxidizing at least a portion of the ethylbenzene to form ethylbenzene hydroperoxide, catalytically reacting the ethylbenzene hydroperoxide with propylene to form propylene oxide and 1-phenyl ethanol, and dehydrating the 1-phenyl ethanol to produce styrene monomer;
b) dehydrogenating at least a portion of the ethylbenzene to produce styrene monomer; or
c) a combination thereof.

Embodiment A16. The process any one of Embodiments A1-A15, further comprising:
a) recovering the propylene from the third intermediate product or the fourth intermediate product;
b) catalytically reacting the propylene with ethylbenzene hydroperoxide to form propylene oxide and 1-phenyl ethanol; and
c) dehydrating the 1-phenyl ethanol to produce styrene monomer.

Embodiment A17. The process any one of Embodiments A1-A16, further comprising:
a) adding at least a portion of the first residual fraction to the first reaction zone as additional feed;
b) sending at least a portion of the first residual fraction outside the process for further processing; or
c) a combination thereof.

Embodiment A18. The process any one of Embodiments A1-A18, further comprising:
a) adding at least a portion of the second residual fraction to the first reaction zone as additional feed;
b) adding at least a portion of the second residual fraction to the second reaction zone as additional feed;
c) sending at least a portion of the first residual fraction outside the process for further processing; or
d) a combination thereof.

Embodiment B1. A method for converting a fuel oil to propylene comprising: (a) providing a fuel oil comprising a plurality of aromatic compounds comprising: (i) from about 1.0 wt % to about 2.0 wt % ethylbenzene; and (ii) from about 1.0 wt % to about 2.0 wt % styrene, wherein the fuel oil comprises no more than 1.0 wt % benzene and 0.2 wt % propylene; and (b) catalytically cracking the fuel oil in the presence of a catalyst to produce a cracked product comprising: (iii) from about 10 wt % to about 19 wt % propylene; and (iv) from about 36 wt % to about 50 wt % benzene.

Embodiment B2. The method of Embodiment B1, wherein the method is further characterized by one or more of the following additional elements:
a) Element 1: catalytic cracking the fuel oil occurs at a temperature greater than about 380° C.;
b) Element 2: catalytic cracking the fuel oil occurs at a temperature greater than about 480° C.;
c) Element 3: catalytic cracking the fuel oil occurs at a temperature greater than about 580° C.;
d) Element 4: the catalyst is a zeolite; and
e) Element 5: the catalyst is a H-ZSM-5 zeolite.

Embodiment C1. A method for converting a fuel oil to propylene comprising:
a) providing a fuel oil comprising a plurality of aromatic compounds comprising:
   i) from about 1.0 wt % to about 2.0 wt % ethylbenzene; and
   ii) from about 1.0 wt % to about 2.0 wt % styrene;
   wherein the fuel oil comprises no more than 1.0 wt % benzene and 0.2 wt % propylene; and
b) catalytically cracking the fuel oil in the presence of a catalyst to produce a cracked product comprising:
   i) from about 10 wt % to about 19 wt % propylene; and
   ii) from about 36 wt % to about 50 wt % benzene.

Embodiment C2. The method of Embodiment C1, wherein catalytic cracking the fuel oil occurs at a temperature greater than about 380° C., greater than about 480° C., or greater than about 580° C.

Embodiment C3. The method of Embodiment C1 or C2, wherein the catalyst is a zeolite.

Embodiment C4. The method of Embodiment C3, wherein the zeolite is a H-ZSM-5 zeolite.

The presently disclosed processes are exemplified in the examples below. These examples are included to demonstrate embodiments of the appended claims. However, these are exemplary only, and the invention can be broadly applied to embodiments not demonstrated in the examples. Those of skill in the art should appreciate that many changes can be made in the specific embodiments which are disclosed and still obtain a like or similar result without departing from the spirit and scope of the disclosure herein. In no way should the following examples be read to limit, or to define, the scope of the appended claims.

### EXAMPLES

The following examples are included to demonstrate embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Background

Characterization of liquid products: The liquid products from the two traps were characterized by Gas Chromatography (GC) and proton NMR (¹H NMR).

The GC analysis of the liquid product for each run was performed using an Agilent 7890 GC (Agilent Technologies, Santa Clara, CA) equipped with a standard non-polar column and a flame ionization detector. For the GC data, the weight percent for x<nC7 (having boiling point<98° C. named LF1), nC7<x<nC11 (having boiling point 98° C.<BP<203° C. named LF2), nC12<x<nC28 (having boiling point 203° C.<BP<434° C. named LF3), x>C28 (having boiling point>434° C. named LF4) were used to characterize the liquid product.

NMR data were used to characterize the percent of aromatic protons, paraffinic protons and olefinic protons in the liquid product. The examples were analyzed with an addition of CDCl3 (0.6 g of depolymerize polymer/metal oxide mixture with 0.4 g of CDCl3). The data were collected on a Bruker AV500 MHz NMR spectrometer (Bruker Corporation, Billerica, MA) at 25° C. with a 5 mm Prodigy probe. One dimension 1H NMR data were processed using TOPSPIN® software (Bruker) with an exponential line broadening window function. Quantitative measurements were performed with a 15 second relaxation delay, a 30° flip angle pulse, and 32 scans to facilitate accurate integrals. The spectral integrations for aromatic olefinic, and paraffinic protons were obtained and used to quantify relative ratios of these protons.

### FTIR Process

FTIR spectroscopy was carried out as follows. An iS50 FTIR spectrometer equipped with a DTGS detector from Thermo Scientific was used for FTIR spectral acquisition. The sample compartment was fitted with an attenuated total reflectance (ATR) accessory. The ATR cell was obtained from Pike Technologies and was equipped with a 3-bounce zinc selenide (ZnSe) crystal. A sample volume of about 0.05 mLs was required for analysis. Those familiar with FTIR spectroscopy will recognize that when multiple spectra are overlaid in the same plot, these spectra can be shown with similar baselines or offset baselines. This choice is purely for optimal viewing of changes occurring and does not impact peak height or peak absorbance measurements as such measurements are made from the baseline to the top of the peak.

### Thermal and Catalytic Cracking Studies

In each test, approximately 0.5-1 mg of a waste feed material and ˜50 mg of catalyst was added to a Frontier Lab Tandem Micro-Reactor system attached to an Agilent GC/MS with a flow of 200 ml/min of helium through the headspace above the polymer recyclate. Each sample was heated to 500 or 550° C. for 1 minute. The pyrolysate gas passed through a quartz tube containing an optional catalyst maintained at a specified cracking temperature. Reaction product gas was cryo-trapped at the head of the GC/MS column for subsequent separation and identification.

### Determination of Catalytic Pyrolysis Reaction Product

The composition of the reaction product was measured with Pyrolysis Gas Chromatography-Mass Spectrometry (Pyrolysis-GC/MS-FID) analysis using a Rx-3050TR Tandem micro-Reactor, Frontier labs, Fukushima, JP; and an Agilent 8890 GC equipped with a flame ionization detector (FID) and 5977B MSD and UA1 (30 m×250 μm×2 μm) GC column, Agilent Technologies, Santa Clara, California. The GC is also equipped with a splitter to split the column effluent between the FID and MSD.

GC/MS, or gas chromatography-mass spectrometry, was used to identify the composition of the reaction product stream. Of particular interest are the ethane and ethylene (C2), propane and propylene (C3), mixed butanes and butenes (C4), and benzene/toluene/xylenes (BTX). The quantification was done using the FID signal from each experiment. (

### Thermal Pyrolysis

Table 1 shows results of non-catalytic pyrolysis tests on residual fuel oil and polystyrene pyrolysis oil heavies. Results show similar increases in styrene and ethylbenzene after thermal pyrolysis at 500° C.

TABLE 1

| | | Polystyrene |
|---|---|---|
| | Residual Fuel | Pyrolysis Oil |
| Product | Oil | Heavies |
| Styrene (wt %) | 67.5 | 79.3 |
| Toluene (wt %) | 4.6 | 7.5 |
| A-Me-Styrene (wt %) | 2.7 | 4.6 |
| Ethylbenzene (wt %) | 23.5 | 1.7 |
| Benzeneacetaldehyde (wt %) | <0.1 | 1.8 |
| Acetophenone (wt %) | <0.1 | 1.8 |
| 2-Phenelpropenal (wt %) | <0.1 | 1.6 |

### Catalytic Pyrolysis

Table 2 shows results of catalytic pyrolysis tests on residual fuel oil and polystyrene pyrolysis oil heavies. Results show similar increases in styrene and ethylbenzene after catalytic pyrolysis using mordenite at 500° C.

TABLE 2

| | | Polystyrene |
|---|---|---|
| | Residual Fuel | Pyrolysis Oil |
| Product | Oil | Heavies |
| Benzene (wt %) | 60.1 | 62.6 |
| Naphthalene (wt %) | 16.2 | 18.3 |
| C₂^{═} and C₃^{═} (wt %) | 4.8 | 7.1 |
| Toluene (wt %) | 7.9 | 6.0 |
| Me-Naphthalene (wt %) | 3.2 | 2.6 |

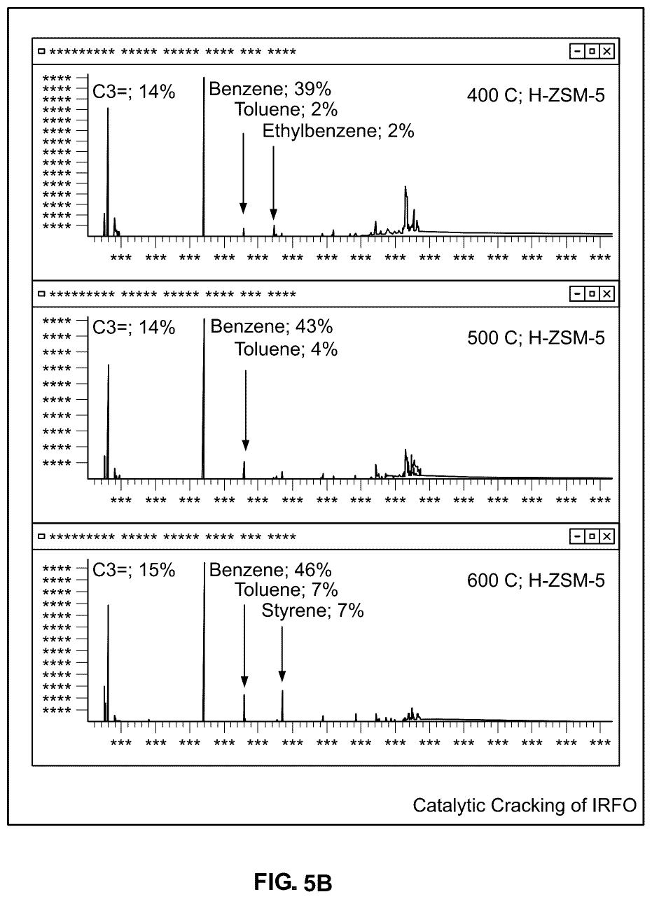

FIG. 5A presents two spectra produced from the fuel oil used in the below Examples. The lower of the two spectra presents a close up view of a portion of the upper spectra. Various peaks are numbered corresponding to different chemical species identified below the spectra. The percentages represent the wt % of the chemical species present in the fuel oil. For example, 1.5 wt % ethylbenzene. FIG. 5B presents three spectra of the product produced by catalytically cracking the fuel oil identified in the spectra of FIG. 5A at three different temperatures: 400° C., 500° C., and 600° C.

### Example 1

A fuel oil having the composition indicated by the spectra shown in FIG. 5A was catalytically cracked at 400° C. in the presence of a H-ZSM-5 catalyst in a Frontier Lab tandem microreactor GC/MS system to produce a cracked product comprising about 14 wt % propylene, about 39 wt % benzene, about 2 wt % toluene, and about 2 wt % ethylbenzene as shown in FIG. 5B.

### Example 2

A fuel oil having the composition indicated by the spectra shown in FIG. 5A was catalytically cracked at 500° C. in the presence of a H-ZSM-5 catalyst in a Frontier Lab tandem microreactor GC/MS system to produce a cracked product comprising about 14 wt % propylene, about 43 wt % benzene, and about 4 wt % toluene as shown in FIG. 5B.

### Example 3

A fuel oil having the composition indicated by the spectra shown in FIG. 5A was catalytically cracked at 600° C. in the presence of a H-ZSM-5 catalyst in a Frontier Lab tandem microreactor GC/MS system to produce a cracked product comprising about 15 wt % propylene, about 46 wt % benzene, about 7 wt % toluene, and about 7 wt % styrene as shown in FIG. 5B.

For the sake of brevity, only certain ranges are explicitly disclosed herein. However, in addition to recited ranges, any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the processes, machines, means, processes, and/or steps described in the specification. As one of the ordinary skill in the art will readily appreciate from the disclosure of the present invention, processes, machines, means, processes, and/or steps, presently existing or later to be developed that perform substantially the same function or achieve substantially the same result as the corresponding embodiments described herein, may be utilized according to the present invention. Accordingly, the appended claims are intended to include within their scope such processes, machines, means, processes, and/or steps.

What is claimed is:

1. A process to upgrade a heavy aromatic waste stream to chemical feedstocks, the process comprising:
a) subjecting the heavy aromatic waste stream to thermal pyrolysis conditions in a first reaction zone to produce a first intermediate product;
b) optionally subjecting the first intermediate product to separation conditions to produce a second intermediate product and a first residual fraction;
c) subjecting the first intermediate product or the second intermediate product to catalytic pyrolysis conditions in a second reaction zone to produce a third intermediate product comprising styrene, benzene, ethylene, propylene, or a combination thereof; and
d) optionally subjecting the third intermediate product to separation conditions to produce a fourth intermediate product comprising styrene, benzene, ethylene, propylene, or a combination thereof and a second residual fraction.

2. The process of claim 1, wherein:
a) the heavy aromatic waste stream has a first styrene content;
b) the first intermediate product has a second styrene content, and the second styrene content is greater than the first styrene content;
c) the second intermediate product has a third styrene content, and the third styrene content is greater than the second styrene content;
d) the third intermediate product has a fourth styrene content, and the fourth styrene content is greater than the third styrene content; and
e) the fourth intermediate product has a fifth styrene content, and the fifth styrene content is greater than the fourth styrene content.

3. The process of claim 1, wherein:
a) the heavy aromatic waste stream has a first benzene content;
b) the first intermediate product has a second benzene content, and the second benzene content is greater than the first benzene content;
c) the second intermediate product has a third benzene content, and the third benzene content is greater than the second benzene content;
d) the third intermediate product has a fourth benzene content, and the fourth benzene content is greater than the third benzene content; and
e) the fourth intermediate product has a fifth benzene content, and the fifth benzene content is greater than the fourth benzene content.

4. The process of claim 1, wherein:
a) the heavy aromatic waste stream has a first ethylene content;
b) the first intermediate product has a second ethylene content, and the second ethylene content is greater than the first ethylene content;
c) the second intermediate product has a third ethylene content, and the third ethylene content is greater than the second ethylene content;
d) the third intermediate product has a fourth ethylene content, and the fourth ethylene content is greater than the third ethylene content; and
e) the fourth intermediate product has a fifth ethylene content, and the fifth ethylene content is greater than the fourth ethylene content.

5. The process of claim 1, wherein:
a) the heavy aromatic waste stream has a first propylene content;
b) the first intermediate product has a second propylene content, and the second propylene content is greater than the first propylene content;
c) the second intermediate product has a third propylene content, and the third propylene content is greater than the second propylene content;
d) the third intermediate product has a fourth propylene content, and the fourth propylene content is greater than the third propylene content; and
e) the fourth intermediate product has a fifth propylene content, and the fifth propylene content is greater than the fourth propylene content.

6. The process of claim 1, wherein the thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

7. The process of claim 1, wherein the catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

8. The process of claim 1, wherein the second reaction zone comprises a medium or large pore zeolite or molecular sieve.

9. The process of claim 8, wherein the medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

10. The process of claim 1, further comprising:
a) recovering the propylene from the third intermediate product or the fourth intermediate product;
b) catalytically reacting the propylene with ethylbenzene hydroperoxide to form propylene oxide and 1-phenyl ethanol; and
c) dehydrating the 1-phenyl ethanol to produce styrene monomer.

11. The process of claim 1, further comprising:
a) adding at least a portion of the first residual fraction to the first reaction zone as additional feed;
b) sending at least a portion of the first residual fraction outside the process for further processing; or
c) a combination thereof.

12. The process of claim 1, further comprising:
a) adding at least a portion of the second residual fraction to the first reaction zone as additional feed;
b) adding at least a portion of the second residual fraction to the second reaction zone as additional feed;
c) sending at least a portion of the first residual fraction outside the process for further processing; or
d) a combination thereof.
